(19) United States
(12) Patent Application Publication
(10) Pub. No.: US 2025/0201356 A1
(43) Pub. Date: Jun. 19, 2025

(54) MOLECULAR SIMULATION METHOD AND INFORMATION PROCESSING APPARATUS

(71) Applicant: Fujitsu Limited, Kawasaki-shi (JP)

(72) Inventors: Yasunari HIKIMA, Kawasaki (JP); Yuhei UMEDA, Kawasaki (JP)

(73) Assignee: Fujitsu Limited, Kawasaki-shi (JP)

(21) Appl. No.: 19/065,010

(22) Filed: Feb. 27, 2025

Related U.S. Application Data
(63) Continuation of application No. PCT/JP2022/036624, filed on Sep. 30, 2022.

(51) Int. Cl. G16C 20/30; G16C 20/70

(52) U.S. Cl. CPC G16C 20/30; G16C 20/70

(57) ABSTRACT

An information processing apparatus estimates, for each of a plurality of interatomic distances, an execution time of a first algorithm that obtains a molecular energy using quantum circuit data. The information processing apparatus determines an interatomic distance group on the basis of a time limit and the execution times. The information processing apparatus obtains a first molecular energy corresponding to a first interatomic distance included in the interatomic distance group, by executing the first algorithm on the first interatomic distance. The information processing apparatus outputs the first molecular energy and a second molecular energy corresponding to a second interatomic distance, obtained by a second algorithm, the second interatomic distance being not included in the interatomic distance group.

### CROSS-REFERENCE TO RELATED APPLICATION

This application is a continuation application of International Application PCT/JP2022/036624 filed on Sep. 30, 2022, which designated the U.S., the entire contents of which are incorporated herein by reference.

### FIELD

The embodiments discussed herein relate to a molecular simulation method and an information processing apparatus.

### BACKGROUND

Computers may perform molecular simulations to analyze the properties of molecules through numerical calculations. Molecular simulations may be used in industrial fields such as material development and pharmaceutical development. Molecular simulations involve quantum chemical calculations that microscopically calculate the energy of a molecule on the basis of the electronic state of the molecule and the Schrödinger equation.

Quantum chemical calculation algorithms include algorithms using quantum circuit data, such as variational quantum eigensolver (VQE). It is also possible to use quantum computers to execute such algorithms using quantum circuit data. In addition, other quantum chemical calculation algorithms include configuration interaction (CI) methods and coupled cluster (CC) methods.

For the configuration interaction methods, a quantum chemical computing device has been proposed that dynamically selects some molecular orbitals from a plurality of molecular orbitals of a molecule and calculates the energy of the molecule on the basis of electron configurations that are limited to the selected molecular orbitals.

See, for example, International Publication Pamphlet No. WO 2022/097298.

### SUMMARY

According to one aspect, there is provided a non-transitory computer-readable storage medium storing a computer program that causes a computer to perform a process including: estimating an execution time of a first algorithm for each of a plurality of interatomic distances based on molecular information specifying a molecule to be analyzed, the first algorithm being configured to obtain a molecular energy using quantum circuit data; determining, from among the plurality of interatomic distances, an interatomic distance group with respect to which the first algorithm is to be executed, based on a specified time limit and the estimated execution time; obtaining a first molecular energy corresponding to a first interatomic distance included in the determined interatomic distance group, by executing the first algorithm on the first interatomic distance; and outputting the first molecular energy and a second molecular energy corresponding interatomic distance, obtained by a second algorithm different from the first algorithm, the second interatomic being not included in the interatomic distance group among the plurality of interatomic distances.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1 is a view for describing an information processing apparatus according to a first embodiment;

FIG. 2 illustrates an example of the hardware configuration of an information processing apparatus according to a second embodiment;

FIG. 3 is a graph representing an example of a potential energy curve;

FIG. 4 is a graph representing an example of the accuracy of classical algorithms and a quantum algorithm;

FIG. 5 illustrates an example of a method of estimating the execution time of a VQE job;

FIG. 6 illustrates example results of estimating the execution times and costs of VQE jobs;

FIG. 7 illustrates examples of the relationship between user-specified upper limits and the number of VQE jobs;

FIG. 8 is a graph representing an example of the relationship between interatomic distance and iteration count of a classical algorithm;

FIG. 9 illustrates an example of adding a VQE job;

FIG. 10 is a block diagram illustrating an example of the functions of the information processing apparatus;

FIG. 11 is a flowchart illustrating an example procedure for quantum chemical calculation; and

FIG. 12 is a flowchart illustrating an example procedure for execution time estimation.

### DESCRIPTION OF EMBODIMENTS

In some cases, a computer may calculate the molecular energy of a molecule while changing the interatomic distance of two target atoms and analyze the relationship between the interatomic distance and the molecular energy. For example, the computer generates a potential energy curve (PEC) representing the relationship between the interatomic distance and the ground-state energy of the molecule.

A quantum chemical calculation algorithm has trade-off relationship between accuracy and execution time. In addition, the computer is not always allowed to spend a huge amount of time on quantum chemical calculation, and a time limit may be imposed by a user or another. Therefore, it is important to determine how to select an algorithm for a plurality of interatomic distances, from the perspective of efficiency in the quantum chemical calculation.

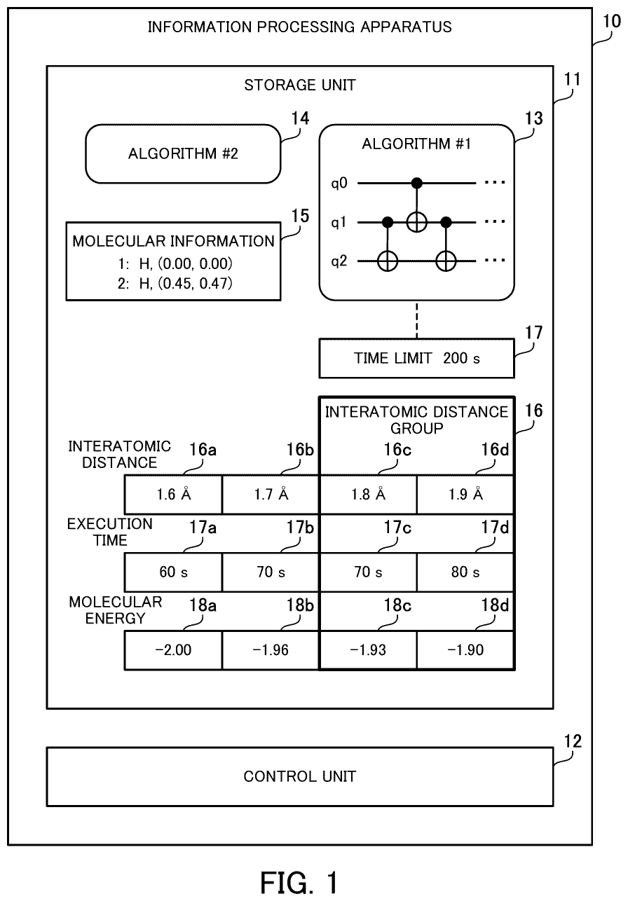

Hereinafter, embodiments will be described with reference to the accompanying drawings. A first embodiment will now be described. FIG. 1 is a view for describing an information processing apparatus according to the first embodiment. The information processing apparatus 10 of the first embodiment carries out molecular simulation through quantum chemical calculation. The information processing apparatus 10 calculates a plurality of molecular energies corresponding to a plurality of interatomic distances and outputs information associating each interatomic distance with its corresponding molecular energy. For example, the information processing apparatus 10 generates and outputs a potential energy curve. The information processing apparatus 10 may be a client device or a server device. The information processing apparatus 10 may also be referred to as a computer, a molecular simulation device, a quantum chemical calculation device, or an algorithm selection device.

The information processing apparatus 10 includes a storage unit 11 and a control unit 12. The storage unit 11 may be a volatile semiconductor memory, such as random access memory (RAM), or a non-volatile storage device, such as a hard disk drive (HDD) or flash memory.

The control unit 12 is, for example, a processor such as a central processing unit (CPU), a graphics processing unit (GPU), or a digital signal processor (DSP). Note that the control unit 12 may include an application specific integrated circuit (ASIC), a field programmable gate array (FPGA), or another electronic circuit. For example, the processor executes programs stored in a memory such as RAM (which may be the storage unit 11). The processor may be referred to as processor circuitry. A set of processors may be referred to as a multiprocessor or simply a “processor.” Different processors may perform different ones of a plurality of processes that will be described later.

The storage unit 11 stores molecular information 15 specifying a molecule to be analyzed. The molecular information 15 defines a molecular structure by, for example, specifying the types and coordinates of each atom in the molecule. In addition, the storage unit 11 stores a plurality of interatomic distances for which molecular energies are to be calculated. An interatomic distance refers to the distance between two target atoms in the molecule. The distance is, for example, the Euclidean distance. The molecular energy is, for example, the ground-state energy that is obtained when the molecule is in a stable state. A change in the interatomic distance results in a change in the molecular energy.

As an example, the storage unit 11 stores interatomic distances 16a, 16b, 16c, and 16d. The interatomic distance 16b is larger than the interatomic distance 16a, and the interatomic distance 16c is larger than the interatomic distance 16b, and the interatomic distance 16d is larger than the interatomic distance 16c. In addition, the storage unit 11 stores a time limit 17. The time limit 17 is an upper limit of time that is imposed on the quantum chemical calculation, and may be specified by a user. For example, the time limit 17 is an upper limit of the total execution time to calculate all the molecular energies corresponding to the plurality of interatomic distances.

The control unit 12 calculates and outputs the plurality of molecular energies corresponding to the plurality of interatomic distances. In this calculation, the control unit 12 selectively uses one of the algorithms 13 and 14 depending on an interatomic distance. The algorithms 13 and 14 are different algorithms for quantum chemical calculation, and are designed to calculate molecular energies on the basis of the molecular information 15. In this connection, the information processing apparatus 10 may cause another information processing apparatus to execute the algorithm 13, instead of executing the algorithm 13 by itself. Likewise, the information processing apparatus 10 may cause e another information processing apparatus to execute the algorithm 14, instead of executing the algorithm 14 by itself.

The algorithm 13 calculates molecular energies using quantum circuit data. The algorithm 13 is, for example, a quantum algorithm such as a variational quantum eigensolver (VQE). The quantum circuit data is a quantum computation model that defines gate operations to be applied to qubits. The algorithm 13 may be executed by a gate-based quantum computer. Alternatively, the algorithm 13 may be executed by a von Neumann-type classical computer using software that simulates operations of a quantum computer.

For example, the quantum circuit data defines an ansatz circuit and a measurement circuit. The ansatz circuit generates a quantum state using one or more qubits, and is generated based on basis functions that approximate the wave function of the Schrödinger equation. The measurement circuit measures a molecular energy from the quantum state, and is generated based on a Hamiltonian of the Schrödinger equation determined by the type of the molecule.

For example, the algorithm 13 generates a quantum state and measures a molecular energy a plurality of times with respect to a given electronic configuration, and calculates an expected value of the molecular energy for the electronic configuration. The algorithm 13 iteratively calculates the expected value of the molecular energy while changing the electronic configuration, in order to search for the minimum molecular energy. The algorithm 13 outputs the minimum molecular energy as the ground-state energy. As the interatomic distance is larger, the effects of outer molecular orbitals become more significant, so that a longer time may be needed to find the minimum molecular energy. Therefore, the execution time of the algorithm 13 that is taken until the molecular energy converges may increase.

The algorithm 14 calculates molecular energy in a different manner from the algorithm 13. For example, the algorithm 14 is a classical algorithm that does not use quantum circuit data, and is designed to be executed by a classical computer. The algorithm 14 may be a configuration interaction method such as configuration interaction singles and doubles (CISD), or a coupled cluster method such as coupled cluster singles and doubles (CCSD) or CCSD and triples (CCSD(T)).

It is preferred that the computational complexity and execution time of the algorithm 14 be significantly less than those of the algorithm 13. However, the accuracy of the algorithm 14 may be lower than that of the algorithm 13. Especially, as an interatomic distance increases, the effects of higher-order electronic excitations become more significant, which may reduce the accuracy of the algorithm 14.

For example, the algorithm 14 generates fixed computational formulas based on basis functions that approximate a wave function, and calculates the molecular energy for a given electronic configuration. To reduce the computational complexity, the algorithm 14 may disregard triple- and higher order-electronic excitations, or quadruple- and higher order-electronic excitations. The algorithm 14 iteratively calculates the molecular energy while changing the electronic configuration, in order to search for the minimum molecular energy. The algorithm 14 then outputs the minimum molecular energy as the ground-state energy. As the interatomic distance increases, the accuracy is lower, and therefore the iteration count needed until the molecular energy converges may increase.

In order to selectively use the algorithms 13 and 14, the control unit 12 estimates the execution time of the algorithm 13 for each of the plurality of interatomic distances on the basis of the molecular information 15, without actually executing the algorithm 13. As an example, the control unit 12 estimates execution times 17a, 17b, 17c, and 17d corresponding respectively to the interatomic distances 16a, 16b, 16c, and 16d.

For example, for each of the plurality of interatomic distances, the control unit 12 executes the algorithm 14, and then estimates the execution time of the algorithm 13 on the basis of the execution result of the algorithm 14. The execution result of the algorithm 14 may be the iteration count of the algorithm 14. In addition, the control unit 12 may use feature values of the quantum circuit data used by the algorithm 13 in the estimation of the execution time. Furthermore, the control unit 12 may use a trained machine learning model to estimate the execution time of the algorithm 13. The machine learning model may be a regression model.

The control unit 12 determines an interatomic distance group 16 with respect to which the algorithm 13 is to be executed, from among the plurality of interatomic distances on the basis of the estimated execution times for the individual interatomic distances and the time limit 17. For example, the control unit 12 classifies as many interatomic distances as possible into the interatomic distance group 16, provided that the total estimated execution time of the interatomic distances included in the interatomic distance group 16 does not exceed the time limit 17. For example, the control unit 12 classifies interatomic distances into the interatomic distance group 16, preferentially in order starting with the largest interatomic distance. Alternatively, for example, the control unit 12 classifies interatomic distances into the interatomic distance group 16, preferentially in order starting with the interatomic distance with the highest iteration count of the algorithm 14. As an example, the control unit 12 classifies the interatomic distances 16c and 16d into the interatomic distance group 16.

The control unit 12 executes the algorithm 13 on each interatomic distance included in the determined interatomic distance group 16 to calculate their corresponding molecular energies. As an example, the control unit 12 calculates molecular energies 18c and 18d corresponding respectively to the interatomic distances 16c and 16d.

In addition, the control unit 12 executes the algorithm 14 on each interatomic distance that is not included in the interatomic distance group 16, to calculate their corresponding molecular energies. As an example, the control unit 12 calculates molecular energies 18a and 18b corresponding respectively to the interatomic distances 16a and 16b. Note, however, that the molecular energies 18a and 18b may have already been calculated by executing the algorithm 14 for estimating the execution times 17a and 17b. In this case, the control unit 12 does not need to calculate the molecular energies 18a and 18b again.

Then, the control unit 12 outputs the molecular energies 18a and 18b corresponding to the interatomic distances 16a and 16b, calculated by the algorithm 14, and the molecular energies 18c and 18d corresponding to the interatomic distances 16c and 16d, calculated by the algorithm 13. For example, the control unit 12 outputs a potential energy curve associating each interatomic distance with its corresponding molecular energy. The control unit 12 may store the calculated molecular energies in a non-volatile storage device, display them on a display device, or send them to another information processing apparatus.

As described above, the information processing apparatus 10 of the first embodiment estimates the execution time of the algorithm 13 that uses quantum circuit data, for each of the plurality of interatomic distances on the basis of the molecular information 15. The information processing apparatus 10 determines the interatomic distance group 16 with respect to which the algorithm 13 is to be executed, on the basis of the specified time limit 17 and the estimated execution times. The information processing apparatus 10 executes the algorithm 13 on each interatomic distance included in the interatomic distance group 16 to calculate their corresponding molecular energies. The information processing apparatus 10 outputs the molecular energies corresponding to the interatomic distances included in the interatomic distance group 16, calculated by the algorithm 13, and the molecular energies corresponding to the other interatomic distances, calculated by the algorithm 14.

As described above, the information processing apparatus 10 is able to selectively use the algorithms 13 and 14, taking trade-off between accuracy and execution time into account, so as to satisfy the specified time limit 17. As a result, it is achieved to calculate the plurality of molecular energies corresponding to the plurality of interatomic distances efficiently.

In this connection, the information processing apparatus 10 may estimate the execution cost of the algorithm 13 for each of the plurality of interatomic distances, and may determine the interatomic distance group 16, further taking a specified cost limit and the estimated execution costs into account. This makes it possible to streamline the calculation of molecular energies while taking the execution costs such as costs into account.

In addition, the information processing apparatus 10 may estimate the execution time of the algorithm 13 on the basis of an execution result of the algorithm 14. This contributes to improving the accuracy of estimating the execution time. In addition, the information processing apparatus 10 may determine the interatomic distance group 16, provided that the total execution time of the interatomic distance group 16 does not exceed the time limit 17. This allows the information processing apparatus 10 to output the molecular energies by the user-desired time.

Furthermore, the information processing apparatus 10 may classify interatomic distances into the interatomic distance group 16, preferentially in order starting with the largest interatomic distance. This improves the accuracy, preferentially for interatomic distances in which the accuracy of the algorithm 14 is likely to be low. In addition, the information processing apparatus 10 may classify interatomic distances into the interatomic distance group 16, preferentially in order starting with the interatomic distance with the highest iteration count of the algorithm 14. This makes it possible to execute the algorithm 13 to recalculate molecular energies, preferentially for interatomic distances in which the accuracy of the algorithm 14 is low, and therefore to improve the accuracy for these interatomic distances.

In addition, in the case where the algorithm 13 is completed before an estimated execution time elapses, the information processing apparatus 10 may additionally execute the algorithm 13 on one or more interatomic distances that are not included in the interatomic distance group 16. This allows the information processing apparatus 10 to use available computing resources, in order to improve the accuracy of calculating molecular energies. The algorithm 13 may be a VQE, and the algorithm 14 may be a coupled cluster method. This achieves a balance between accuracy and execution time for the plurality of interatomic distances as a whole.

A second embodiment will now be described. The information processing apparatus 100 of the second embodiment generates a potential energy curve representing the relationship between the distance of two target atoms and the ground-state energy of a molecule through quantum chemical calculation. The information processing apparatus 100 is able to execute a plurality of algorithms. Note, however, that some or all of the algorithms may be executed by another information processing apparatus. The other information processing apparatus may be a quantum computer. The information processing apparatus 100 may be a client device or a server device. The information processing apparatus 100 may be installed in a data center or provided in a cloud system. The cloud system may receive a job request for quantum chemical calculation over a network and return a generated potential energy curve. The information processing apparatus 100 may be referred to as a computer, a molecular simulation device, or a quantum chemical calculation device. The information processing apparatus 100 corresponds to the information processing apparatus 10 of the first embodiment.

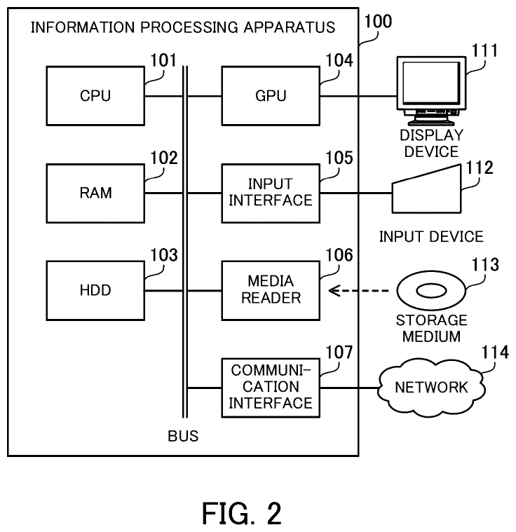

FIG. 2 illustrates an example of the hardware configuration of the information processing apparatus according to the second embodiment. The information processing apparatus 100 includes a CPU 101, a RAM 102, an HDD 103, a GPU 104, an input interface 105, a media reader 106, and a communication interface 107, which are connected to a bus. The CPU 101 corresponds to the control unit 12 of the first embodiment. The RAM 102 or HDD 103 corresponds to the storage unit 11 of the first embodiment.

The CPU 101 is a processor that executes program commands. The CPU 101 loads programs and data from the HDD 103 to the RAM 102 and executes the programs. The information processing apparatus 100 may be provided with a plurality of processors.

The RAM 102 is a volatile semiconductor memory that temporarily stores programs being executed by the CPU 101 and data being used by the CPU 101 in processing. The information processing apparatus 100 may be provided with a different type of volatile memory than RAM.

The HDD 103 is a non-volatile storage device that stores software programs such as an operating system (OS), middleware, and application software, and data. The information processing apparatus 100 may be provided with another type of non-volatile storage device such as flash memory or solid state drive (SSD).

The GPU 104 performs image processing in collaboration with the CPU 101 and outputs images to a display device 111 connected to the information processing apparatus 100. Examples of the display device 111 include a cathode ray tube (CRT) display, a liquid crystal display, an organic electro-luminescence (EL) display, and a projector. Another type of output device such as a printer may be connected to the information processing apparatus 100.

The GPU 104 may be used as a general-purpose computing on graphics processing unit (GPGPU). The GPU 104 is able to execute programs in accordance with instructions from the CPU 101. The information processing apparatus 100 may be provided with a volatile semiconductor memory other than the RAM 102 as a GPU memory.

The input interface 105 receives an input signal from an input device 112 connected to the information processing apparatus 100. Examples of the input device 112 include a mouse, a touch panel, and a keyboard. A plurality of input devices may be connected to the information processing apparatus 100.

The media reader 106 is a reading device that reads programs and data from a storage medium 113. Examples of the storage medium 113 include a magnetic disk, an optical disc, and a semiconductor memory. Magnetic disks include flexible disks (FDs) and HDDs. Optical discs include compact discs (CDs) and digital versatile discs (DVDs). The media reader 106 copies the programs and data from the storage medium 113 to another storage medium such as the RAM 102 or the HDD 103. The read programs may be executed by the CPU 101.

The storage medium 113 may be a portable storage medium. The storage medium 113 may be used to distribute the programs and data. The storage medium 113 and HDD 103 may be referred to as computer-readable storage media. The communication interface 107 communicates with other information processing apparatuses over a network 114. The communication interface 107 may be a wired communication interface that is connected to a wired communication device such as a switch or a router, or may be a wireless communication interface that is connected to a wireless communication device such as a base station or an access point.

The following describes a quantum chemical calculation and a solution-finding algorithm for the quantum chemical calculation. The quantum chemical calculation is one type of molecular simulation and analyzes molecular structures and intermolecular interactions from electronic states. The quantum chemical calculation may be used to assist material development and pharmaceutical development. The quantum chemical calculation is microscopic molecular simulation, and achieves high analytical accuracy but has high computational load.

A quantum chemical calculation solves the Schrödinger equation, HΨ=EΨ, where H denotes a Hamiltonian, Ψ denotes a wave function, and E denotes energy. The Hamiltonian H depends on a target molecular structure. The wave function Y describes an electronic eigenstate, and the energy E is an eigenenergy corresponding to Ψ. The quantum chemical calculation calculates the ground-state energy when the molecular structure is stable. However, it is difficult to directly solve the Schrödinger equation.

To address this, in the quantum chemical calculation, the wave function Ψ is expressed as basis functions. Each basis function is represented as a linear combination of known functions, and each term in the basis function corresponds to a molecular orbital. The molecular orbital is a location where any one of the electrons in the molecule may enter. The quantum chemical calculation receives the specification of molecular information indicating the positions of the plurality of atoms in the molecule, a solution-finding algorithm, and basis functions, and calculates the ground-state energy on the basis of the specified information. Note, however, that there is no need to specify the solution-finding algorithm in the second embodiment. The information processing apparatus 100 generates a potential energy curve through the quantum chemical calculation.

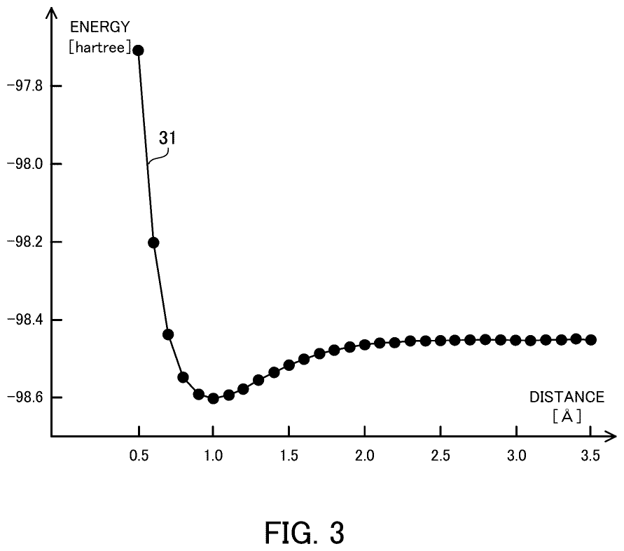

FIG. 3 is a graph representing an example of a potential energy curve. The curve 31 is a potential energy curve. The potential energy curve represents potential energies corresponding to different interatomic distances. A potential energy represents the energy that the molecule would have when each atom is assumed to be at rest. The horizontal axis of the potential energy curve represents interatomic distance, whereas the vertical axis thereof represents ground-state energy.

The unit of distance is, for example, angstrom (Å). The unit of energy is, for example, hartree. An energy is calculated for each of a plurality of discrete distances within a certain range. The plurality of distances may be equally spaced. For example, the energy may be calculated at 0.1 Å intervals from 0.5 Å to 3.5 Å. The potential energy curve is generated by plotting the calculated energies and connecting the plotted points with a line. The minimum point on the potential energy curve may represent the most stable state of the molecule. The maximum point on the potential energy curve may represent a transition state of the molecule.

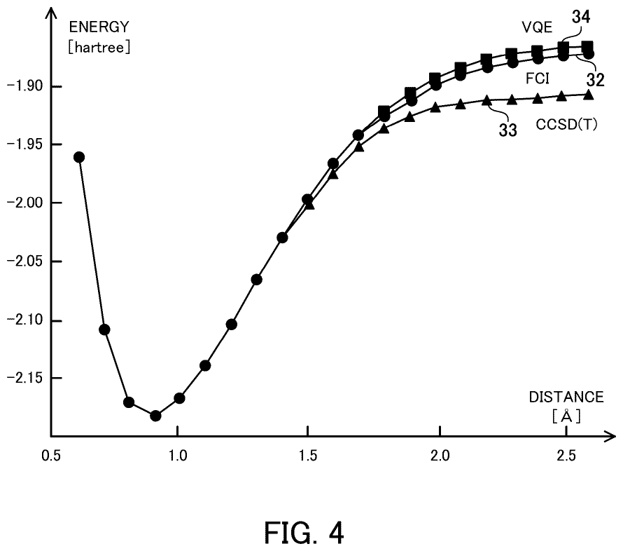

FIG. 4 is a graph representing examples of the accuracy of classical algorithms and a quantum algorithm. The second embodiment provides the case of selectively using the CCSD(T) and the VQE as quantum chemical calculation algorithms. In this connection, the CISD or CCSD may be used instead of the CCSD(T). FIG. 4 also includes full configuration interaction (FCI) as an algorithm with significantly high accuracy.

A curve 32 is a potential energy curve generated using the FCI only. A curve 33 is a potential energy curve generated using the CCSD(T) only. A curve 34 is a potential energy curve generated using the VQE only.

The FCI is a classical algorithm designed to run on a classical computer. The FCI finds the exact energy solution on the basis of specified molecular information and basis functions. Therefore, the FCI exhibits high solution accuracy but has a long execution time. The FCI has a computational complexity of an order that is the factorial of the number of molecular orbitals. Therefore, it is difficult to calculate the energies of large-scale molecules using the FCI. Since the FCI has a property of finding exact solutions, the energies calculated by the FCI may be interpreted as correct energies.

The CCSD(T) is a classical algorithm designed to run on a classical computer. The CCSD(T) finds an approximate energy solution on the basis of specified molecular information and basis functions. Therefore, the CCSD(T) exhibits lower solution accuracy than the FCI and has a shorter execution time than the FCI. The CCSD(T) has a computational complexity of an order that is the seventh power of the number of molecular orbitals. In this connection, the CCSD exhibits lower solution accuracy than the CCSD(T) and has a shorter execution time than the CCSD(T).

The CCSD(T) precisely calculates the effects of single- and double-electron excitations on energy and obtains the effect of triple-electron excitation through perturbations, as an electron state. On the other hand, the CCSD(T) disregards the effects of quadruple- and higher order-electron excitations. The CCSD(T) iteratively calculates energy while changing the electron configuration, in order to search for the minimum energy. The CCSD(T) performs the iterative process until the calculated energy converges. For example, the CCSD(T) compares the most recent energy with its immediately previous energy, and completes the iterative process when the difference between these two energies falls below a threshold.

In the case where an interatomic distance is small, the CCSD(T) mostly calculates a relatively good approximate solution, compared with the FCI. In the case where an interatomic distance is large, on the other hand, the CCSD(T) may calculate an approximate solution with low accuracy. This is because, in the case where the interatomic distance is large, the effects of outer molecular orbitals on the energy are large, and the CCSD(T), which disregards the effects of quadruple- and higher order-electron excitations, has a large error in the approximate solution. In addition, in the case where the accuracy of the final output energy is low, the CCSD(T) tends to have an increased iteration count until convergence. This is because the approximate solution may continue to fluctuate near the correct value even through iterations of the iterative process and thus may fail to stably converge toward the correct value.

The VQE is a quantum algorithm designed to run on a gate-based quantum computer. Alternatively, the VQE may be able to run on a classical computer with a quantum simulator. The quantum simulator simulates operations of a quantum computer by software. In this case, each time a single qubit is added to the classical computer, its memory usage and computational complexity get double. The second embodiment provides the case of executing the VQE using such a quantum simulator. In terms of solution accuracy and execution time, the VQE is in the middle between the FCI and the CCSD(T). That is to say, the VQE exhibits lower solution accuracy than the FCI and higher solution accuracy than the CCSD(T). The VQE has a shorter execution time than the FCI and a longer execution time than the CCSD(T).

The VQE generates a quantum circuit that generates a quantum state using a plurality of qubits on the basis of specified basis functions. This quantum circuit may be called an ansatz circuit. In addition, the VQE generates a quantum circuit that measures energy from the quantum state on the basis of the Hamiltonian corresponding to specified molecular information. This quantum circuit may be called a measurement circuit. Each quantum circuit is a quantum computation model represented as a combination of quantum gates. In a quantum computer, a quantum circuit is implemented with physical qubits. In a quantum simulator, pseudo-qubit data is stored in a memory, and pseudo-quantum gate operations are implemented using classical programs.

The VQE uses the ansatz circuit to generate a quantum state and then uses the measurement circuit to measure energy. Each measurement has been affected by noise and fluctuations. Therefore, for the same electron configuration, the VQE generates a quantum state and measures energy a plurality of times, and then calculates the average of these measurements as an expected value of energy. The VQE adjusts the parameter values used for generating the quantum state so as to make the expected value of energy smaller. An adjustment in the parameter values is equivalent to a change in the electron configuration. By iteratively performing the above process, the VQE searches for the ground-state energy. For example, the VQE iteratively performs the above process until the expected value of energy converges.

A “classical computer” is, for example, a von Neumann-type computer, which is contrasted with a “quantum computer.” A “classical algorithm” is, for example, an algorithm that is contrasted with a “quantum algorithm” and does not use quantum circuits.

As seen in the curves 33 and 34, the accuracy of the CCSD(T) is noticeably lower than that of the VQE, depending on the distance. On the other hand, the execution time of the VQE is noticeably longer than that of the CCSD(T). For example, the execution time of the VQE may exceed 1000 times that of the CCSD(T). In view of this, the information processing apparatus 100 is unable to disregard the execution time and cost needed to generate a potential energy curve. The information processing apparatus 100 may be requested to generate the potential energy curve within upper limits specified by the user. For example, the cost is a cost that the user bears for using the information processing apparatus 100.

Therefore, the information processing apparatus 100 performs automatic algorithm selection in order to generate as accurate a potential energy curve as possible within the user-specified upper execution time limit and cost limit. The algorithm selection is made on a distance-by-distance basis.

In the second embodiment, the information processing apparatus 100 first executes the CCSD(T) on all distances. The information processing apparatus 100 then estimates the execution time and cost of the VQE for each distance on the basis of the execution results of the CCSD(T). Then, the information processing apparatus 100 selects distances on which the VQE is to be additionally executed, on the basis of the estimated execution times, estimated costs, upper execution time limit, and upper cost limit. In generating a potential energy curve, the information processing apparatus 100 employs the energies calculated by the VQE for the selected distances and employs the energies calculated by the CCSD(T) for the other distances.

The following describes the estimation of execution time for the VQE. The information processing apparatus 100 estimates the execution time of the VQE using machine learning models trained in advance. The machine learning models may be referred to as estimators. The machine learning models of the second embodiment are Gaussian process regression models generated by a Gaussian process. In addition, the machine learning of training these machine learning models may be performed by the information processing apparatus 100 or another information processing apparatus.

The machine learning models include a time model that estimates the execution time per iteration of the VQE and an iteration model that estimates the iteration count of the VQE. The execution time per iteration corresponds to the time needed to calculate an expected value of energy for a single electron configuration. The iteration count corresponds to the number of attempts in each of which the electron configuration is changed. The estimated value of the execution time of the VQE is calculated as the product of the execution time estimated by the time model and the iteration count estimated by the iteration model.

Note that the actual iteration count may fluctuate due to randomness, and there is therefore an upside risk of the actual iteration count exceeding the expected value. In addition, due to a small amount of training data, uncertainty may arise in the estimation result obtained by the iteration model. To address these, the information processing apparatus 100 may use an iteration model that outputs an iteration count exceeding the expected value, with taking at least one of the randomness and uncertainty into account. The following describes an example of the machine learning models using mathematical formulae.

First, the time model that estimates an execution time per iteration will be described. The explanatory variable of the time model is a three-dimensional vector x defined by Formula (1), where q denotes the number of qubits, d denotes the depth of an ansatz circuit, and l denotes the number of terms in a Hamiltonian. The depth of the ansatz circuit refers to the number of stages of the quantum gates arranged in series. The number of terms in the Hamiltonian refers to the number of terms obtained by decomposing the Hamiltonian into a sum of Pauli matrices.

$$x = ( q , d , ℓ ) \qquad ( 1 )$$

The time model, which calculates the expected value of the execution time per iteration, is defined as Formula (2), for example. In Formula (2), y is a response variable representing the execution time per iteration, and n denotes the number of records in training data. Training data to be used for training the time model includes n records, each containing a value of the explanatory variable and a value of the response variable as a pair, represented as (x₁, y₁), . . . , (xₙ, yₙ).

$$y = ( {y}_{1} , … , {y}_{n} ) {( {K}_{n} + λ {I}_{n} )}^{- 1} {k}_{n} ( x ) \qquad ( 2 )$$

Denote k as the kernel of a Gaussian process. The kernel k is a function that defines the similarity between vectors. Examples of the kernel k include a radial basis function (RBF) kernel and a Matern kernel. In Formula (2), Kₙ is an n×n square matrix generated from values of the explanatory variable in the training data. The element in the i-th row and j-th column of the matrix Kₙ is k(xᵢ, xⱼ). The matrix Kₙ represents the similarity between two values of the explanatory variable in the training data. In is an n×n identity matrix. kₙ(x) is a column vector whose i-th row element is k(xᵢ, x). kₙ(x) indicates the similarity between a certain vector x and each of n values of the explanatory variable included in the training data. λ is a constant greater than zero.

Considering the risk of an actual execution time per iteration deviating from an expected value, the information processing apparatus 100 is able to use a time model that takes robustness against that risk into account. First, as seen in Formula (3), a conditional value at risk (CVaR) is defined for the execution time per iteration. In Formula (3), α is a constant greater than zero and less than or equal to one. Ψν(y) and U are defined as in Formula (4).

$${CVaR}_{n , α} ( x ; {y}_{1} , … , {y}_{n} ) = {max}_{v ∈ U} \{ v - \frac{1}{α} ( {ψ}_{v} ( {y}_{1} ) , … , {ψ}_{v} ( {y}_{n} ) ) {( {K}_{n} + λ {I}_{n} )}^{- 1} {k}_{n} ( x ) \} \qquad ( 3 )$$

$${ψ}_{v} ( y ) = max ( v - y , 0 ) , U = \{ {y}_{1} , … , {y}_{n} \} \qquad ( 4 )$$

For example, the time model that takes the robustness into account is defined as Formula (5) using CVaR of Formula (3). The estimated value calculated by Formula (5) reflects the upside risk of the execution time per iteration and is therefore expected to be greater than the expected value calculated by Formula (2). Denoting ρ as a distribution of the vector x and denoting F as a cumulative distribution function corresponding to the distribution ρ, Formula (5) provides an estimated value of Formula (6).

$$- {CVaR}_{n , 1 - α} ( x ; - {y}_{1} , … , - {y}_{n} ) \qquad ( 5 )$$

$${E}_{y \sim ρ ( x )} [ y \vert y \geq {F}^{- 1} ( α ) ] \qquad ( 6 )$$

In addition, further considering uncertainty in the estimation of the time model due to insufficient training data, the information processing apparatus 100 is able to use a time model that takes both the robustness and the uncertainty into account. First, as seen in Formula (7), σₙ(x) is defined for the execution time per iteration. In Formula (7), kₙ^{T}(x) is the transpose matrix of kₙ(x).

$${σ}_{n} ( x ) = \sqrt{k ( x , x ) - {k}_{n}^{T} ( x ) {K}_{n}^{- 1} {k}_{n} ( x )} \qquad ( 7 )$$

For example, the time model that takes both the robustness and the uncertainty into account is defined as Formula (8) using σₙ(x) of Formula (7). In Formula (8), β is a positive constant. The estimated value calculated by Formula (8) reflects an additional upside risk of the execution time per iteration and is therefore greater than the estimated value calculated by Formula (5).

$$- {CVaR}_{n , 1 - α} ( x ; - {y}_{1} , … , - {y}_{n} ) + β {σ}_{n} ( x ) \qquad ( 8 )$$

Next, the iteration model that estimates an iteration count will be described. The basic structure of the iteration model is the same as that of the time model. Note, however, that the explanatory variable and response variable in the iteration model have different definitions from those in the time model. The explanatory variable of the iteration model is a two-dimensional vector z as defined by Formula (9), where m denotes an iteration count of a classical algorithm and s denotes an interatomic distance.

The second embodiment uses the CCSD(T) as a classical algorithm. Alternatively, the CISD or CCSD may be used as the classical algorithm. Note that, in a broad sense, the term “CCSD” may be interpreted as including both the narrow definitions of the CCSD and CCSD(T).

$$z = ( m , s ) \qquad ( 9 )$$

For example, the iteration model that estimates an iteration count is defined as Formula (10), where w is a response variable representing the iteration count of the VQE. Training data to be used for training the iteration model includes n records, each containing a value of the explanatory variable and a value of the response variable as a pair, represented as (z₁, z₁), . . . , (zₙ, wₙ).

$$w = ( {w}_{1} , … , {w}_{n} ) {( {L}_{n} + λ {I}_{n} )}^{- 1} {ℓ}_{n} ( z ) \qquad ( 10 )$$

In Formula (10), denote 1 as the kernel of a Gaussian process. Lₙ is an n×n square matrix generated from values of the explanatory variable included in the training data. The element in the i-th row and j-th column of the matrix Lₙ is l (zᵢ, zⱼ). lₙ(z) is a column vector whose i-th row element is l(zᵢ, z). λ is a constant greater than zero.

As with the time model, considering the risk of an actual iteration count deviating from an expected value, the information processing apparatus 100 is able to use an iteration model that takes robustness against that risk into account. For example, the iteration model that takes the robustness into account is defined as Formula (11) using CVAR of Formula (3). Here, in Formulae (3) and (4), x is replaced with z, y is replaced with w, Kₙ is replaced with Lₙ, and kₙ is replaced with lₙ.

$$- {CVaR}_{n , 1 - α} ( z ; - {w}_{1} , … , - {w}_{n} ) \qquad ( 11 )$$

In addition, further considering uncertainty in the estimation of the iteration model due to insufficient training data, the information processing apparatus 100 is able to use an iteration model that takes both the robustness and the uncertainty into account. For example, the iteration model that takes both the robustness and the uncertainty into account is defined as Formula (12) using Formula (7). Here, in Formula (7), x is replaced with z, Kₙ is replaced with Lₙ, and kₙ is replaced with lₙ.

$$- {CVaR}_{n , 1 - α} ( z ; - {w}_{1} , … , - {w}_{n} ) + β {σ}_{n} ( z ) \qquad ( 12 )$$

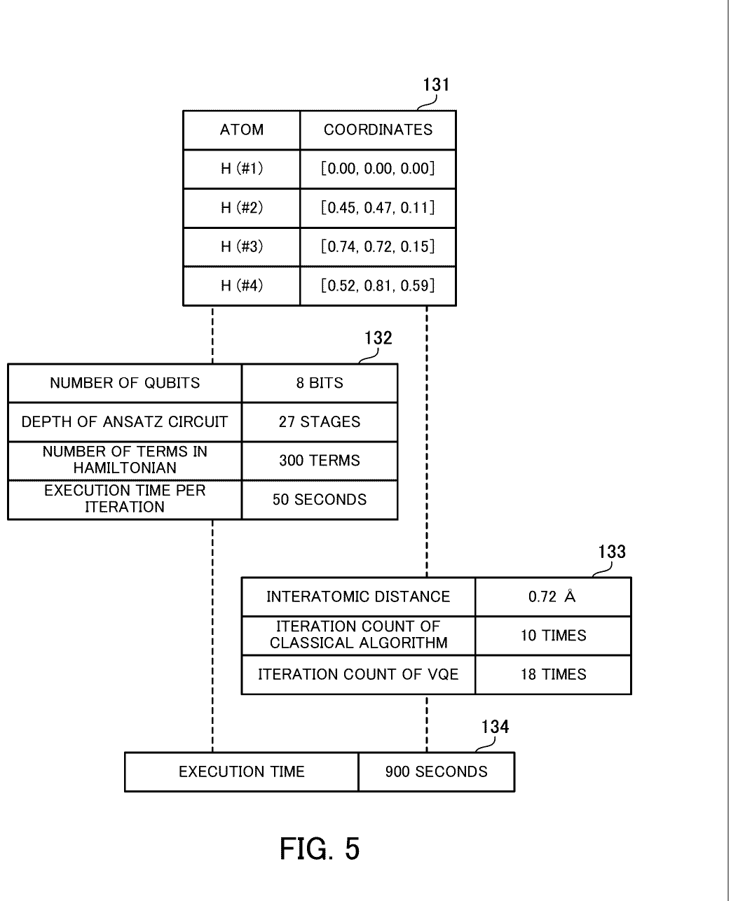

FIG. 5 illustrates an example of a method of estimating the execution time of a VQE job. In the following, a process of calculating an energy corresponding to a single distance using the VQE may be referred to as a VQE job. The information processing apparatus 100 obtains data 131 on a molecule to be analyzed. The data 131 indicates the type and coordinates of each atom in the molecule. At the time of machine learning, n sets of data that is like the data 131 are used as sample data.

The information processing apparatus 100 generates data 132 from the data 131. The data 132 includes the number of qubits, the depth of an ansatz circuit, the number of terms in a Hamiltonian, and an execution time per iteration. The number of qubits, the depth of the ansatz circuit, and the number of terms in the Hamiltonian are input data to the time model and are calculated from the data 131 through preprocessing of the VQE. The execution time per iteration is output data from the time model.

At the time of the machine learning, n sets of data that is like the data 132 are used as training data to be used for training the time model. In this case, the execution time per iteration corresponds to teacher data and is measured by executing the VQE for sample molecular information.

In addition, the information processing apparatus 100 generates data 133 from the data 131. The data 133 includes an interatomic distance, an iteration count of a classical algorithm, and an iteration count of the VQE. The interatomic distance and the iteration count of the classical algorithm are input data to the iteration model. The iteration count of the classical algorithm is measured by executing the classical algorithm on the basis of the data 131. The iteration count of the VQE is output data from the iteration model.

At the time of the machine learning, n sets of data that is like the data 133 are used as training data to be used for training the iteration model. In this case, the iteration count of the VQE corresponds to teacher data and is measured by executing the VQE.

The information processing apparatus 100 generates data 134 from the data 132 and 133. The data 134 includes an estimated value of the execution time of the VQE. The execution time is calculated as the product of the execution time per iteration included in the data 132 and the iteration count of the VQE included in the data 133. One or both of the execution time per iteration output from the time model and the iteration count of the VQE output from the iteration model may each represent an expected value, an estimated value obtained taking robustness into account, or an estimated value obtained taking both robustness and uncertainty into account. The information processing apparatus 100 may switch the estimated value type in accordance with user instruction.

After completing the estimation of the execution time for each distance, the information processing apparatus 100 estimates a cost for each distance on the basis of the corresponding estimated execution time. The cost is proportional to the execution time. For example, the estimated cost is calculated as the product of a coefficient, the estimated execution time, and the number of computing nodes used. In the case where the unit of execution time is second and the unit of cost is yen, for example, the coefficient is 0.1. In the following, it is assumed for simple description that the user uses only one computing node.

The following describes how to select distances as targets for VQE jobs. The information processing apparatus 100 executes VQE jobs such that the total estimated execution time is less than or equal to a user-specified upper execution time limit and the total estimated cost is less than or equal to a user-specified upper cost limit. Assume that the execution time of the classical algorithm is negligibly small.

At this time, the information processing apparatus 100 selects distances, preferentially in order starting with the one that achieves the highest accuracy improvement effect through the VQE, i.e., in order starting the one with the lowest accuracy under the classical algorithm. As described above, the classical algorithm has a higher risk of accuracy deterioration as the distance increases. Therefore, one distance selection method is to select as many distances as possible in order starting with the largest distance.

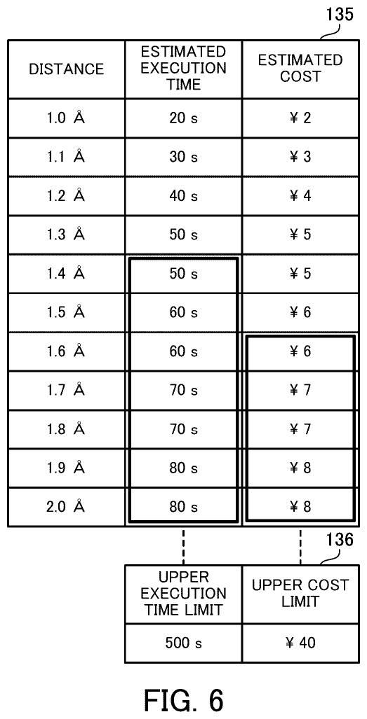

FIG. 6 illustrates example results of estimating the execution times and costs of VQE jobs. In this example, eleven distances in a range of 1.0 Å to 2.0 Å are target candidates for VQE jobs. A table 135 associates each distance with an estimated execution time and an estimated cost. The estimated execution time is preferably an estimated value obtained taking robustness and uncertainty into account, in order to reduce the risk of exceeding an upper execution time limit or an upper cost limit due to the execution time of a VQE job exceeding the corresponding estimated execution time.

The estimated execution time and estimated cost of the VQE job with a distance of 1.0 Å are 20 seconds and 2 yen, respectively. The estimated execution time and estimated cost of the VQE job with a distance of 1.1 Å are 30 seconds and 3 yen, respectively. The estimated execution time and estimated cost of the VQE job with a distance of 1.2 Å are 40 seconds and 4 yen, respectively. The estimated execution times and estimated costs of the VQE jobs with distances of 1.3 Å and 1.4 Å are 50 seconds and 5 yen, respectively. The estimated execution times and estimated costs of the VQE jobs with distances of 1.5 Å and 1.6 Å are 60 seconds and 6 yen, respectively. The estimated execution times and estimated costs of the VQE jobs with distances of 1.7 Å and 1.8 Å are 70 seconds and 7 yen, respectively. The estimated execution times and estimated costs of the VQE jobs with distances of 1.9 Å and 2.0 Å are 80 seconds and 8 yen, respectively.

Data 136 indicates a user-specified upper execution time limit and a user-specified upper cost limit. Here, the upper execution time limit is 500 seconds and the upper cost limit is 40 yen. In the case of selecting as many distances as possible in order starting with the largest distance such as to ensure that the upper execution time limit is not exceeded, the distances 1.4 Å to 2.0 Å are selected. In the case of selecting as many distances as possible in order starting with the largest distance such as to ensure that the upper cost limit is not exceeded, the distances 1.6 Å to 2.0 Å are selected. Therefore, the selection of the distances 1.6 Å to 2.0 Å complies with both the upper execution time limit and the upper cost limit.

In this distance selection method, as a higher execution time limit and a higher cost limit are specified by the user, more distances are selected in the descending order as targets for VQE jobs. Depending on the specified upper execution time limit and upper cost limit, no distances may be selected as targets for VQE jobs or all distances may be selected as targets for VQE jobs.

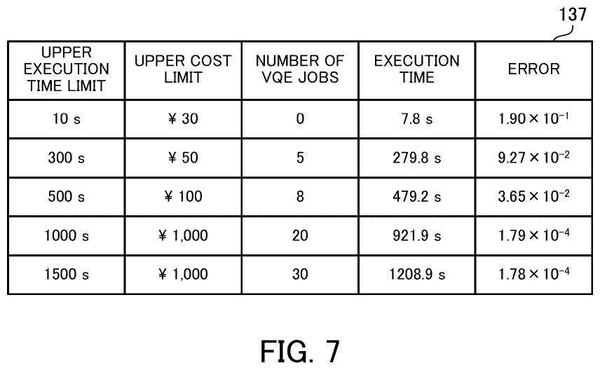

FIG. 7 illustrates examples of the relationship between user-specified upper limits and the number of VQE jobs. A table 137 describes the relationship between upper execution time limit, upper cost limit, the number of VQE jobs, execution time, and error. The upper execution time limit and upper cost limit are specified by the user. The number of VQE jobs is the number of distances selected as targets for VQE execution. The execution time is a measured value of the total execution time of the VQE. The error is an error in the overall potential energy curve, e.g., the difference from the energy calculated by the FCI.

These examples correspond to the potential energy curve of FIG. 3. In the case where the upper execution time limit is 10 seconds and the upper cost limit is 30 yen, there is not a single distance to be selected as targets for VQE jobs. In the case where the upper execution time limit is 300 seconds and the upper cost limit is 50 yen, there are five distances as targets for VQE jobs. In the case where the upper execution time limit is 500 seconds and the upper cost limit is 100 yen, there are eight distances as targets for VQE jobs. In the case where the upper execution time limit is 1000 seconds and the upper cost limit is 1000 yen, there are 20 distances as targets for VQE jobs. In the case where the upper execution time limit is 1500 seconds and the upper cost limit is 1000 yen, there are 30 distances as targets for VQE jobs.

As described above, as a higher upper execution time limit and a higher upper cost limit are specified, more distances are selected. As a result, as a higher upper execution time limit and a higher upper cost limit are specified, the user has a longer waiting time, but the potential energy curve will be more accurate.

The following describes another distance selection method. As described earlier, as a calculated energy has lower accuracy, the classical algorithm has an increased iteration count. Therefore, it is conceivable that the information processing apparatus 100 employs a method of selecting distances, preferentially in order starting with the one with the highest iteration count of the classical algorithm.

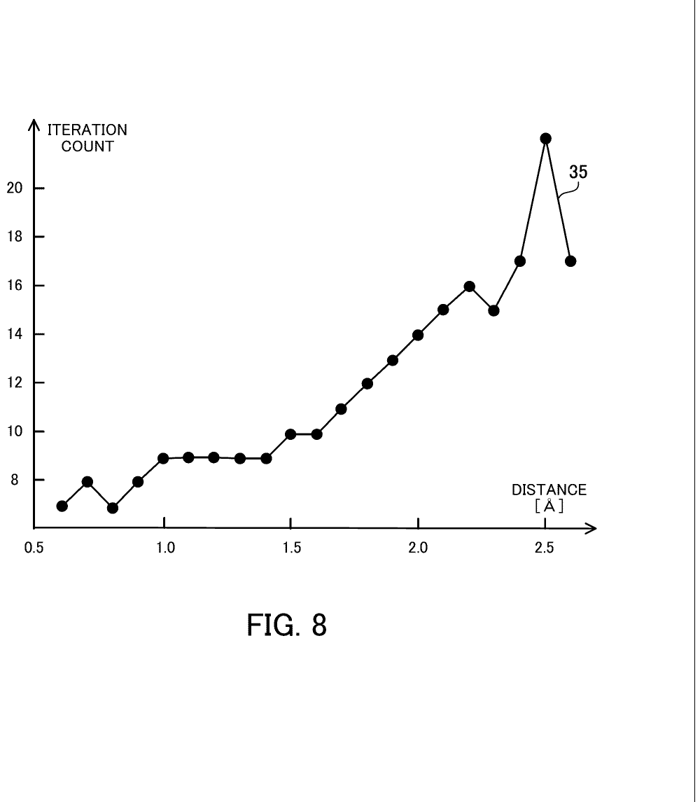

FIG. 8 is a graph representing an example of the relationship between interatomic distance and iteration count of a classical algorithm. A curve 35 represents the relationship between the interatomic distance and the iteration count of the CCSD(T). The information processing apparatus 100 may select as many distances as possible, preferentially in order starting with the one with the highest iteration count, such as to ensure that an upper execution time limit and an upper cost limit are not exceeded. Instead of selecting as many distances as possible, the information processing apparatus 100 may select distances corresponding to iteration counts exceeding a threshold.

Also, instead of selecting as many distances as possible in the descending order, the information processing apparatus 100 may analyze the sequence of the iteration counts of the classical algorithm to detect a boundary distance at which the accuracy of energy begins to drop sharply, and select distances after the boundary distance.

For example, the information processing apparatus 100 scans the iteration counts in ascending order of distance and performs the least-squares method on a fixed number (e.g., five) of most recent distances and iteration counts to calculate the slope of a fitted line segment. The information processing apparatus 100 checks changes in the slope of the line segment in ascending order of distance, and when detecting that the slope increases a certain number of times (e.g., three times) in a row, the information processing apparatus 100 determines that the iteration count has begun to increase sharply and detects the distance of that time as the boundary distance. The information processing apparatus 100 then selects the distances after the boundary distance as targets for VQE jobs.

The following describes the scheduling of VQE jobs. After selecting distances as targets for VQE execution, the information processing apparatus 100 allocates a computing resource to the VQE job corresponding to each distance. In the case where the user uses a single computing node, two or more VQE jobs corresponding to two or more selected distances are sequentially executed on the computing node.

At this time, the information processing apparatus 100 determines the start time of each VQE job on the basis of the estimated execution times for the individual distances. In order to reduce the risk of a VQE job not completing before the start time of the following VQE job, the estimated execution times referenced in the scheduling are preferably estimated values obtained taking robustness and uncertainty into account.

Note, however, that an estimated execution time obtained taking robustness and uncertainty into account is an estimated value that is larger than an expected value. Therefore, if a VQE job is completed unexpectedly earlier than expected, a long idle time may occur. In this case, the information processing apparatus 100 additionally executes the VQE for some of the distances that have not been selected as targets for VQE execution, using the idle time of the computing node.

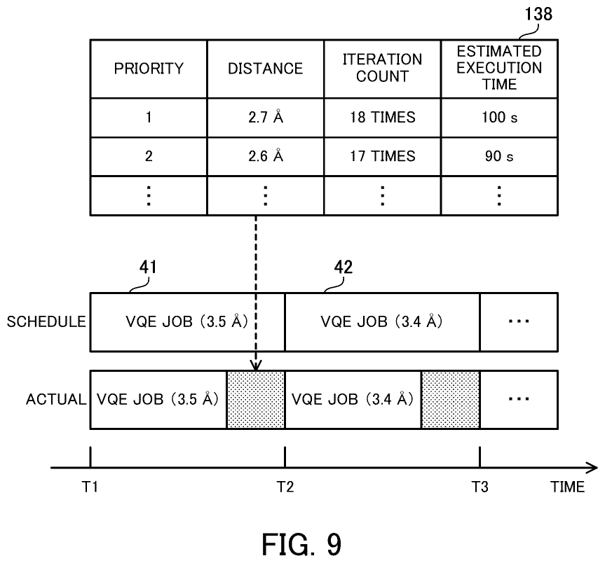

FIG. 9 illustrates an example of adding a VQE job. A VQE job 41 calculates an energy corresponding to a distance 3.5 Å. A VQE job 42 calculates an energy corresponding to a distance 3.4 Å. The start time of the VQE job 41 is time T1 and the scheduled end time thereof is time T2. For example, the time T2 is obtained by adding the estimated execution time for the distance 3.5 Å to the time T1. The start time of the VQE job 42 is the time T2 and the scheduled end time thereof is T3. For example, the time T3 is calculated by adding the estimated execution time for the distance 3.4 Å to the time T2.

Each of the above estimated execution times is an estimated value obtained taking robustness and uncertainty into account. Therefore, the VQE job 41 may be completed well before the time T2. In this case, the information processing apparatus 100 additionally selects any one of the distances that have not been selected for the VQE execution, and executes the additional VQE job using the idle time until the time T2.

A table 138 contains distances that have not been selected as targets for VQE execution. The table 138 associates priority, distance, iteration count, and estimated execution time with each other. The iteration count here is the number of iterations of the classical algorithm. The estimated execution time here is the execution time of the VQE estimated by the above-described method. The priority indicates a descending order of accuracy improvement effect achieved by the VQE. For example, the priority indicates a descending order of distance or a descending order of iteration count.

When completing the VQE job 41, the information processing apparatus 100 calculates an idle time until the time T2 at which the VQE job 42 starts. The information processing apparatus 100 searches the table 138 in order of priority, to find a distance whose estimated execution time is less than or equal to the idle time. That is, the information processing apparatus 100 additionally selects the distance with the highest priority among distances whose estimated execution times are less than or equal to the idle time as a target for a VQE job. The information processing apparatus 100 executes the VQE job corresponding to the additionally selected distance in the idle time.

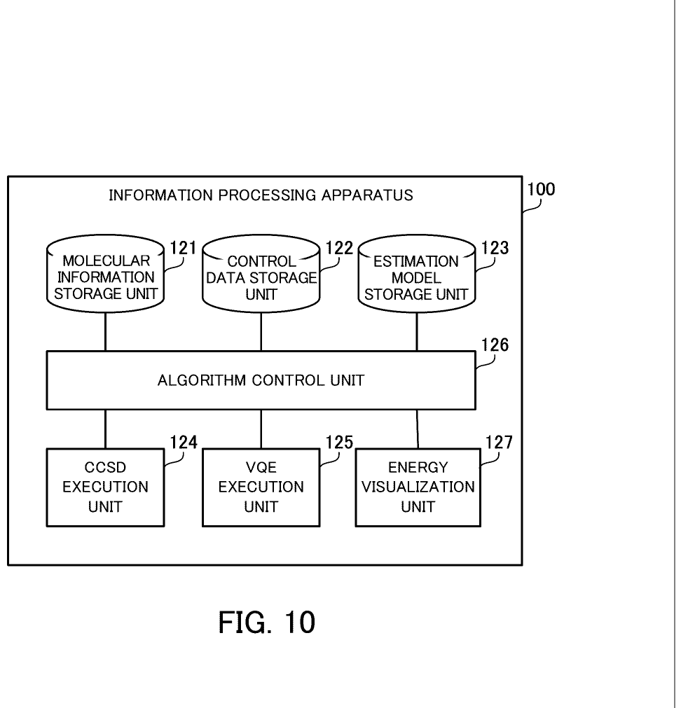

The following describes the functions and processing procedures of the information processing apparatus 100. FIG. 10 is a block diagram illustrating an example of the functions of the information processing apparatus. The information processing apparatus includes a molecular information storage unit 121, a control data storage unit 122, and an estimation model storage unit 123. These storage units are implemented by using, for example, the RAM 102 or HDD 103.

In addition, the information processing apparatus 100 includes a CCSD execution unit 124, a VQE execution unit 125, an algorithm control unit 126, and an energy visualization unit 127. These processing units are implemented by using, for example, the CPU 101 and programs. In this connection, one or both of the CCSD execution unit 124 and the VQE execution unit 125 may be performed by another information processing apparatus.

The molecular information storage unit 121 stores molecular information. The molecular information includes the types and position coordinates of atoms included in a molecule to be simulated. The position coordinates of each atom are adjusted according to the distance between two target atoms. In addition, the molecular information storage unit 121 stores user-specified basis functions. In general, the basis functions are selected from a group of known basis functions by the user according to the type of the molecule and the purpose of the molecular simulation.

The control data storage unit 122 stores a plurality of distances to be used for calculating energy. In addition, the control data storage unit 122 stores the energy calculated by a classical algorithm and the iteration count of the classical algorithm with respect to each of the plurality of distances. In addition, the control data storage unit 122 stores the estimated execution time and estimated cost with respect to each of the plurality of distances. In addition, the control data storage unit 122 stores the energy calculated by the VQE with respect to each distance selected as a target for VQE execution.

The estimation model storage unit 123 stores a time model that estimates the execution time per iteration from feature values of a quantum circuit. In addition, the estimation model storage unit 123 stores an iteration model that estimates the iteration count of the VQE on the basis of an interatomic distance and the iteration count of the classical algorithm. The time model and iteration model have been trained by the information processing apparatus 100 or another information processing apparatus.

The CCSD execution unit 124 executes the CCSD(T) on the basis of specified molecular information and basis functions in accordance with an instruction from the algorithm control unit 126. Alternatively, the CCSD execution unit 124 may execute the CCSD. For the molecular information corresponding to each distance, the CCSD execution unit 124 calculates the ground-state energy and outputs it to the algorithm control unit 126. In addition, the CCSD execution unit 124 measures the iteration count and notifies the algorithm control unit 126 of the iteration count.

The VQE execution unit 125 executes the VQE on the basis of specified molecular information and basis functions in accordance with an instruction from the algorithm control unit 126. The VQE execution unit 125 iteratively generates a quantum circuit and measures energy on the basis of the molecular information and basis functions. For the molecular information corresponding to each distance, the VQE execution unit 125 calculates the ground-state energy and outputs it to the algorithm control unit 126.

The algorithm control unit 126 receives the specification of an upper execution time limit and upper cost limit from the user. The algorithm control unit 126 selects distances as targets for VQE execution so as to achieve the highest accuracy within the specified upper execution time limit and upper cost limit.

First, the algorithm control unit 126 causes the CCSD execution unit 124 to calculate energies corresponding to all distances to thereby obtain the energies and the iteration counts of the classical algorithm. The algorithm control unit 126 also causes the VQE execution unit 125 to perform preprocessing to generate a quantum circuit, and obtains feature values of the quantum circuit for all the distances. The algorithm control unit 126 estimates the execution time and cost of the VQE for each distance on the basis of the feature values of the quantum circuit and the iteration count of the classical algorithm using the machine learning models stored in the estimation model storage unit 123.

The algorithm control unit 126 selects distances as targets for VQE execution, according to a certain selection method on the basis of the estimated execution times and estimated costs for the individual distances and user-specified upper execution time limit and upper cost limit. The distance selection method may be specified by the user. The algorithm control unit 126 causes the VQE execution unit 125 to calculate energies for the selected distances.

The energy visualization unit 127 retrieves a plurality of energies corresponding to a plurality of distances from the control data storage unit 122, and generates a potential energy curve by plotting the retrieved energies. At this time, the energy visualization unit 127 uses the energies obtained by the VQE for the distances subjected to the VQE execution, and uses the energies obtained by the classical algorithm for distances that are not subjected to the VQE execution.

The energy visualization unit 127 outputs the generated potential energy curve. The energy visualization unit 127 may store the potential energy curve in a non-volatile storage device, display it on the display device 111, or send it to another information processing apparatus.

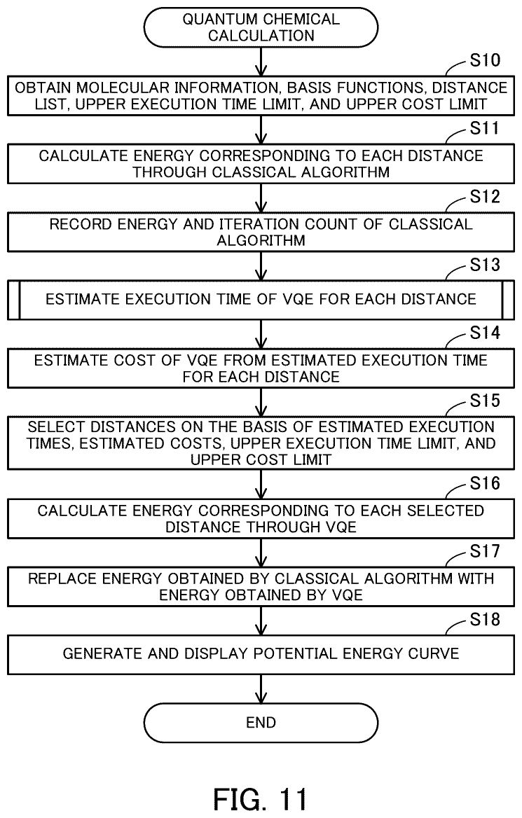

FIG. 11 is a flowchart illustrating an example procedure for quantum chemical calculation. (S10) The algorithm control unit 126 obtains molecular information, basis functions, a distance list, an upper execution time limit, and an upper cost limit. The distance list lists a plurality of distances for which energy is to be calculated.
(S11) The CCSD execution unit 124 calculates an energy corresponding to each of the plurality of distances listed in the distance list, through a classical algorithm such as the CCSD(T). At this time, the CCSD execution unit 124 measures an iteration count incremented until the energy converges.
(S12) The algorithm control unit 126 records the energy and iteration count of the classical algorithm obtained at step S11, with respect to each distance listed in the distance list.
(S13) The algorithm control unit 126 estimates the execution time of the VQE for each distance listed in the distance list. The estimation of the execution time will be described in detail later.
(S14) The algorithm control unit 126 estimates the cost of the VQE from the estimated execution time calculated at step S13, for each distance listed in the distance list.
(S15) The algorithm control unit 126 selects distances as targets for VQE execution from the plurality of distances listed in the distance list, on the basis of the estimated execution times and estimated costs for the individual distances, the upper execution time limit, and the upper cost limit. For example, the algorithm control unit 126 selects as many distances as possible, preferentially in order starting with the largest distance, provided that the upper execution time list and the upper cost limit are not exceeded.
(S16) The VQE execution unit 125 calculates an energy corresponding to each distance selected at step S15, through the VQE.
(S17) The energy visualization unit 127 replaces the energy corresponding to each distance selected at step S15, among the energies calculated by the classical algorithm at step S11, with the energy calculated by the VQE at step S16.
(S18) The energy visualization unit 127 generates a potential energy curve on the basis of the plurality of energies corresponding to the plurality of distances after the replacement of step S17. The energy visualization unit 127 displays the generated potential energy curve.

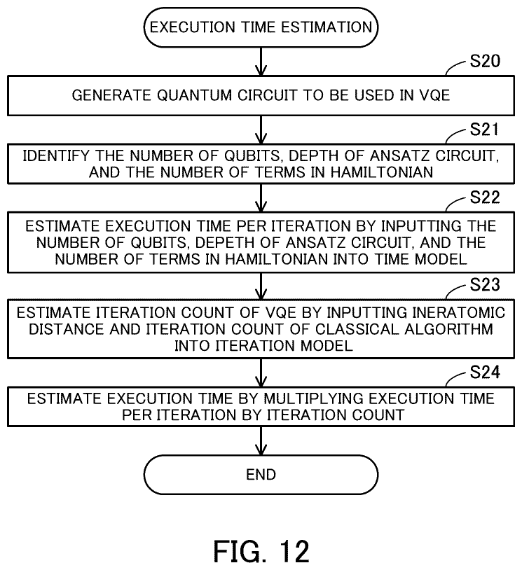

FIG. 12 is a flowchart illustrating an example procedure for execution time estimation. (S20) The VQE execution unit 125 generates a quantum circuit to be used in the VQE, on the basis of molecular information.
(S21) The algorithm control unit 126 identifies the number of qubits, the depth of an ansatz circuit, and the number of terms in a Hamiltonian on the basis of the generated quantum circuit.
(S22) The algorithm control unit 126 inputs the number of qubits, the depth of the ansatz circuit, and the number of terms in the Hamiltonian into a trained time model to thereby estimate the execution time per iteration of the VQE. The execution time per iteration here is an estimated value obtained taking robustness and uncertainty into account, for example.
(S23) The algorithm control unit 126 inputs an interatomic distance and the iteration count of the classical algorithm into a trained iteration model to thereby estimate the iteration count of the VQE. The iteration count here is an estimated value obtained taking robustness and uncertainty into account, for example.
(S24) The algorithm control unit 126 estimates the execution time of the VQE by multiplying the execution time per iteration obtained at step S22 by the iteration count obtained at step S23.

As described above, the information processing apparatus 100 of the second embodiment generates a potential energy curve that represents the relationship between interatomic distance and ground-state energy of a molecule, through quantum chemical calculations. Thereby, the information processing apparatus 100 is able to provide useful information on the properties of the molecule, in order to assist research and development in the fields of material development, pharmaceutical development, and others.

In addition, the information processing apparatus 100 calculates energies using the CCSD(T), which has a shorter execution time, for all interatomic distances, and then recalculates energies using the VQE, which has higher accuracy, for some of the interatomic distances. As a result, the information processing apparatus 100 is able to efficiently generate a potential energy curve while balancing the accuracy and the execution time.

Furthermore, the information processing apparatus 100 estimates the execution time and cost of the VQE for each interatomic distance. The information processing apparatus 100 then selects interatomic distances as targets for VQE execution, provided that the total estimated execution time does not exceed a user-specified upper execution time limit and the total estimated cost does not exceed a user-specified upper cost limit. By doing so, the information processing apparatus 100 is able to generate a potential energy curve with the highest possible accuracy while satisfying the user's requirements.

Furthermore, the information processing apparatus 100 selects interatomic distances, preferentially in order starting with the largest interatomic distance or the interatomic distance with the highest iteration count of the CCSD(T), as targets for VQE execution. This approach enhances the accuracy improvement effect that is achieved through the VQE. In addition, the information processing apparatus 100 estimates the iteration count of the VQE on the basis of the iteration count of the CCSD(T), which improves the accuracy of estimating the execution time. Moreover, the information processing apparatus 100 calculates, as an estimated execution time, an estimated value taking robustness and uncertainty into account. This reduces the risk of the actual total execution time exceeding the upper execution time limit and the risk of the actual total cost exceeding the upper cost limit.

Still further, the information processing apparatus 100 additionally selects interatomic distances as targets for VQE execution in the case where an idle time occurs on a computing node due to the actual execution time of the VQE being shorter than the estimated execution time. In such cases, the information processing apparatus 100 selects interatomic distances, from those whose estimated execution times do not exceed the idle time, preferentially in order starting with the one that achieves the highest accuracy improvement effect through the VQE. By doing so, the information processing apparatus 100 is able to use computing resources effectively and to improve the accuracy of the potential energy curve efficiently.

In one aspect, it is achieved to efficiently calculate molecular energies corresponding to a plurality of interatomic distances.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that various changes, substitutions, and alterations could be made hereto without departing from the spirit and scope of the invention.

What is claimed is:

1. A non-transitory computer-readable storage medium storing a computer program that causes a computer to perform a process comprising:
estimating an execution time of a first algorithm for each plurality of interatomic distances based on molecular information specifying a molecule to be analyzed, the first algorithm being configured to obtain a molecular energy using quantum circuit data;
determining, from among the plurality of interatomic distances, an interatomic distance group with respect to which the first algorithm is to be executed, based on a specified time limit and the estimated execution time;
obtaining a first molecular energy corresponding to a first interatomic distance included in the determined interatomic distance group, by executing the first algorithm on the first interatomic distance; and
outputting the first molecular energy and a second molecular energy corresponding to a second interatomic distance, obtained by a second algorithm different from the first algorithm, the second interatomic distance being not included in the interatomic distance group among the plurality of interatomic distances.

2. The non-transitory computer-readable storage medium according to claim 1, wherein
the estimating of the execution time includes estimating an execution cost of the first algorithm for each of the plurality of interatomic distances, and
the interatomic distance group is determined based on the specified time limit, a specified cost limit, the estimated execution time, and the estimated execution cost.

3. The non-transitory computer-readable storage medium according to claim 1, wherein
the estimating of the execution time includes executing the second algorithm on each of the plurality of interatomic distances based on the molecular information, and
the execution time is estimated based on a result of the executing of the second algorithm.

4. The non-transitory computer-readable storage medium according to claim 1, wherein the interatomic distance group is determined such that a total execution time of the interatomic distance group does not exceed the specified time limit.

5. The non-transitory computer-readable storage medium according to claim 4, wherein the determining of the interatomic distance group includes classifying interatomic distances into the interatomic distance group, preferentially in order starting with a largest interatomic distance among the plurality of interatomic distances.

6. The non-transitory computer-readable storage medium according to claim 4, wherein
the estimating of the execution time includes executing the second algorithm on each of the plurality of interatomic distances based on the molecular information, and
the determining of the interatomic distance group includes classifying interatomic distances into the interatomic distance group, preferentially in order starting with an interatomic distance with a highest iteration count of the second algorithm.

7. The non-transitory computer-readable storage medium according to claim 1, wherein the obtaining of the first molecular energy includes, in response to the first algorithm being completed before the estimated execution time elapses, executing the first algorithm on a third interatomic distance that is not included in the interatomic distance group among the plurality of interatomic distances.

8. The non-transitory computer-readable storage medium according to claim 1, wherein
the first algorithm is a variational quantum eigensolver, and
the second algorithm is a coupled cluster method.

9. A molecular simulation method comprising:
estimating, by a processor, an execution time of a first algorithm for each of a plurality of interatomic distances based on molecular information specifying a molecule to be analyzed, the first algorithm being configured to obtain a molecular energy using quantum circuit data;
determining, by the processor, from among the plurality of interatomic distances, an interatomic distance group with respect to which the first algorithm is to be executed, based on a specified time limit and the estimated execution time;
obtaining, by the processor, a first molecular energy corresponding to a first interatomic distance included in the determined interatomic distance group, by executing the first algorithm on the first interatomic distance; and
outputting, by the processor, the first molecular energy and a second molecular energy corresponding to a second interatomic distance, obtained by a second algorithm different from the first algorithm, the second interatomic distance being not included in the interatomic distance group among the plurality of interatomic distances.

10. An information processing apparatus comprising:
a memory configured to store molecular information specifying a molecule to be analyzed and a plurality of interatomic distances; and
a processor coupled to the memory and the processor configured to:
estimate an execution time of a first algorithm for each of the plurality of interatomic distances based on the molecular information, the first algorithm being configured to obtain a molecular energy using quantum circuit data;
determine, from among the plurality of interatomic distances, an interatomic distance group with respect to which the first algorithm is to be executed, based on a specified time limit and the estimated execution time;
obtain a first molecular energy corresponding to a first interatomic distance included in the determined interatomic distance group, by executing the first algorithm on the first interatomic distance; and
output the first molecular energy and a second molecular energy corresponding to a second interatomic distance, obtained by a second algorithm different from the first algorithm, the second interatomic distance being not included in the interatomic distance group among the plurality of interatomic distances.
